(19) United States
(12) Patent Application Publication
(10) Pub. No.: US 2017/0311901 A1
(43) Pub. Date: Nov. 2, 2017

(54) EXTRACTION OF FEATURES FROM PHYSIOLOGICAL SIGNALS

(71) Applicant: Massachusetts Institute of Technology, Cambridge, MA (US)

(72) Inventors: Mingmin Zhao, Cambridge, MA (US); Fadel Adib, Cambridge, MA (US); Dina Katabi, Cambridge, MA (US)

(21) Appl. No.: 15/490,297

(22) Filed: Apr. 18, 2017

Related U.S. Application Data
(60) Provisional application No. 62/403,808, filed on Oct. 4, 2016.
(60) Provisional application No. 62/323,928, filed on Apr. 18, 2016.

(51) Int. Cl. A61B 5/00; A61B 5/0205; A61B 5/16; A61B 8/08; A61B 5/113; A61B 5/024; A61B 5/05; A61B 5/08; A61B 5/11

(52) U.S. Cl. CPC A61B 5/7278; A61B 8/5223; A61B 5/0205; A61B 5/165; A61B 5/7207; A61B 5/7246; A61B 5/725; A61B 5/7257; A61B 8/0883; A61B 5/113; A61B 5/02416; A61B 5/05; A61B 5/08; A61B 2562/0219; A61B 5/1127; A61B 5/1128; A61B 5/0077; A61B 5/1102

(57) ABSTRACT

A method for determining an emotional state of a subject includes receiving the motion based physiological signal associated with a subject, the motion based physiological signal including a component related to the subject's vital signs, and determining an emotional state of the subject based at least in part on the component related to the subject's vital signs.

### CROSS-REFERENCE TO RELATED APPLICATIONS

This application claims the benefit of the priority dates of U.S. Provisional Application Ser. No. 62/403,808, filed on Oct. 4, 2016 and U.S. Provisional Application Ser. No. 62/323,928, filed on Apr. 18, 2016, the contents of which are incorporated herein by reference.

### STATEMENT AS TO FEDERALLY SPONSORED RESEARCH

This invention was made with Government support under Contract No. FA8721-05-C-0002 awarded by the U.S. Air Force. The Government has certain rights in the invention.

### BACKGROUND

This invention relates to extraction of features from physiological signals, and in particular, signals representing physiological motion.

There is a growing interest in systems capable of inferring the emotions of a subject and, in some cases, reacting to the inferred emotions. Such systems can be used for designing and testing games, movies, advertisement, online content, and human-computer interfaces.

In some examples, systems for inferring the emotions of a subject operate in two stages: In a first stage, they extract emotion related signals (e.g., audio-visual cues or physiological signals) and in a second stage, they feed the emotion related signals into a classifier in to recognize emotions. Existing approaches for extracting emotion-related signals fall into two categories: audiovisual techniques and physiological techniques.

Audiovisual techniques generally rely on facial expressions, speech, and gestures present in an audiovisual recording or stream. Audiovisual approaches do not require users to wear any sensors on their bodies. However, because they rely on outwardly expressed states, they often miss subtle emotions and can be defeated when a subject controls or suppresses outward expression of emotion. Furthermore, many vision-based techniques require the user to face a camera in order for them to operate correctly.

Physiological techniques rely on physiological measurements such as ECG and EEG signals. Physiological measurements are generally more difficult for a subject to control since they are controlled by involuntary activations of the autonomic nervous system (ANS). Existing sensors that can extract these signals require physical contact with a person's body, and therefore interfere with the subject's experience and may affect her emotional state.

Existing approaches for recognizing emotions based on emotion related signals extract emotion-related features from the measured signals and then process the extracted features using a classifier to identify a subject's emotional state. Some existing classification approaches assign each emotion a discrete label (e.g., pleasure, sadness, or anger). Other existing classification approaches use a multidimensional model that expresses emotions in a 2D-plane spanned by valence (i.e., positive vs. negative feeling) and arousal (i.e., calm vs. charged up) axes. For example, anger and sadness are both negative feelings, but anger involves more arousal. Similarly, joy and pleasure are both positive feelings, but the former is associated with excitement whereas the latter refers to a state of contentment.

### SUMMARY

In a general aspect, a method for processing motion based physiological signals representing motion of a subject using signal reflections from the subject includes emitting a radio frequency transmitted signal comprising one or more transmitted signal patterns from a transmitting element. A radio frequency received signal comprising a combination of a number of reflections of the transmitted signal is received at one or more receiving elements, at least some reflections of the number of reflections of the transmitted signal being associated with the subject. Time successive patterns of reflections of the transmitted signal patterns are processed to form the one or more motion based physiological signals including, for at least some reflections of the of the number of reflections, forming a motion based physiological signal representing physiological motion of a subject from a variation over time of the reflection of the transmitted signal in the received signal. Each motion based physiological signal of a subset of the one or more motion based physiological signals is processed to determine a segmentation of a heartbeat component of the motion based physiological signal, the processing including determining the heartbeat component, determining a template time pattern for heartbeats in the heartbeat component, and determining a segmentation of the heartbeat component based on the determined template time pattern.

Aspects may include one or more of the following features.

The transmitted signal may be a frequency modulated continuous wave (FMCW) signal including repetitions of a single signal pattern. The one or more transmitted signal patterns may include one or more pseudo random noise sequences. Determining the heartbeat component may include mitigating an effect of respiration on the motion based physiological signal including determining a second derivative of the motion based physiological signal. Determining the heartbeat component includes mitigating an effect of respiration on the motion based physiological signal may include filtering the motion based physiological signal using a band pass filter. Determining the template time pattern for heartbeats in the heartbeat component and determining the segmentation of the heartbeat component may include jointly optimizing the time pattern for the heartbeats and the segmentation of the heartbeat component.

The method may include determining a cognitive state of the subject based at least in part on the determined segmentation of the heartbeat component of the motion based physiological signal associated with the subject. The cognitive state of the subject may include one or more of a state of confusion, a state of distraction, and a state of attention. The method may include extracting features from the heartbeat components of each of the motion based physiological signals and mapping the extracted features to one or more cardiac functions, the features including as peaks, valleys, of inflection points.

The method may include determining an emotional state of the subject based at least in part on the determined segmentations of the heartbeat components of the motion based physiological signals associated with the subject. Determining the emotional state of the subject may be further based on respiration components of the one or more motion based physiological signals. The method may include determining the respiration components of the one or more motion based physiological signals including applying a low-pass filter to the one or more motion based physiological signals. Determining the emotional state of the subject may include applying an emotion classifier to one or more features determined from the determined segmentations of the heartbeat components of the motion based physiological signals.

Determining the emotional state of the subject may include applying an emotion classifier to one or more features determined from the determined segmentations of the heartbeat components of the motion based physiological signals and to one or more features determined from the respiration components of the one or more motion based physiological signals. The method may include presenting the emotional state in a two-dimensional grid including a first, arousal dimension and a second, valence dimension. The motion based physiological signal may represent physiological motion of a subject from a variation over time of a phase angle of the reflection of the transmitted signal in the received signal.

In another general aspect, a method for determining an emotional state of a subject includes receiving the motion based physiological signal associated with a subject, the motion based physiological signal including a component related to the subject's vital signs, and determining an emotional state of the subject based at least in part on the component related to the subject's vital signs.

Aspects may include one or more of the following features.

The component related to the subject's vital signs may include a periodic component, the method further comprising determining a segmentation of the periodic component. Determining the segmentation of the periodic component may include determining a template time pattern for periods in the periodic component and determining the segmentation of the periodic component based on the determined template time pattern. Determining the emotional state of the subject may be based at least in part on the segmentation of the periodic component. The periodic component may include at least one of a heartbeat component and a respiration component.

Determining the heartbeat component may include determining a second derivative of the motion based physiological signal. The method may include determining the heartbeat component including applying a band-pass filter to the motion based physiological signal. The method may include determining the respiration component including applying a low-pass filter to the motion based physiological signal. Determining the emotional state of the subject may include applying an emotion classifier to one or more features determined from the motion based physiological signal associated with the subject. Determining the emotional state of the subject may include applying an emotion classifier to one or more features determined from the determined segmentation of the periodic component.

The method may include presenting the emotional state in a two-dimensional grid including a first, arousal dimension and a second, valence dimension. The motion based physiological signal associated with the subject may be associated with an accelerometer measurement. The motion based physiological signal associated with the subject may be associated with an ultrasound measurement. The motion based physiological signal associated with the subject may be associated with a radio frequency based measurement. The motion based physiological signal associated with the subject may be associated with a video based measurement.

As is noted above, existing approaches for inferring a person's emotions generally rely on audiovisual cues, such as images and audio clips, or require the person to wear physiological sensors like an ECG monitor. There are limitations associated with both of these existing approaches.

In particular, current audiovisual techniques leverage the outward expression of emotions, but do not measure inner feelings. For example, a person may be happy even if she is not smiling, or smiling even if she is not happy. Also, people differ widely in how expressive they are in showing their inner emotions, which further complicates this problem. Monitoring the physiological signals (e.g., heartbeats) using on-body sensors is an improved approach to measuring a subject's inner emotions since the approach accounts for the interaction between the autonomic nervous system and the heart rhythm. However, using on-body sensors (e.g., ECG monitors) to measure these signals is cumbersome and can interfere with user activity and emotions, making this approach unsuitable for regular usage.

Aspects described herein directly measure physiological signals without requiring a subject to carry sensors on their body and then use the measured physiological signals to estimate an emotion of the subject. In some aspects, the approaches use radio frequency (RF) signals to sense the physiological signals (and the emotions associated with the physiological signals). Specifically, RF reflection signals reflect off the human body and are modulated with bodily movements, including movement associated with breathing and movement associated with heartbeats.

If the individual heartbeats of the heartbeat component of the RF reflection signal can be extracted, minute variations in the length and/or shape of the individual beats are used to estimate the subject's emotion. However, there are a number of challenges associated with extracting individual heartbeats from the RF reflection signals. For example, RF reflection signals are modulated by both the subject's breathing and the subject's heartbeats, with the impact of breathing typically being orders of magnitude larger than that of the heartbeats such that the breathing related motion masks the individual heartbeats. To separate breathing from heart rate, past systems operate over multiple seconds in the frequency domain, forgoing the ability to measure the beat-to-beat variability.

Furthermore, heartbeat-related features (generally referred to as ‘heartbeats’ herein) in the RF reflection signal lack the sharp peaks which characterize the ECG signal, making it harder to accurately identify beat boundaries.

Finally, the difference in inter-beat intervals (IBI) is only a few tens of milliseconds. Thus, individual beats have to be segmented to within a few milliseconds. Obtaining such accuracy is particularly difficult in the absence of sharp features that identify the beginning or end of a heartbeat.

Aspects address these challenges to enable a wireless system that performs emotion recognition using RF reflections off a person's body. Aspects utilize an algorithm for extracting individual heartbeats and the variations between the individual heartbeats from RF reflection signals. In some aspects the algorithm first mitigates the impact of breathing in the RF reflection signals. In some examples, the mitigation mechanism is based on the recognition that, while chest displacement due to the inhale-exhale process is orders of magnitude larger than the minute vibrations caused by heartbeats, the acceleration of motion due to breathing is significantly less than the acceleration of motion due to heartbeats. That is, breathing is usually slow and steady while a heartbeat involves rapid contraction of cardiac muscles at a localized instance in time. Thus, aspects operate on the acceleration of RF reflection signals to dampen the breathing signal and emphasize the heartbeats.

Aspects then segment the RF reflection signal into individual heartbeats. In contrast to the ECG signal which has a known expected shape, the shape of heartbeats in RF reflection signals is unknown and varies depending on the subject's body and exact posture with respect to the device. Thus, aspects are required to learn the beat shape as segmentation occurs. To do so, a joint optimization algorithm iterates between two sub-problems: the first sub-problem learns a template of the heartbeat given a particular segmentation, while the second finds the segmentation that maximizes resemblance to the learned template. The optimization algorithm continues iterating between the two sub-problems until it converges to an optimal beat template and an optimal segmentation that maximizes resemblance to the template.

The segmentation takes into account that beats can shrink and expand and hence vary in beat length. Thus, the algorithm finds the beat segmentation that maximizes the similarity in the morphology of a heartbeat signal across consecutive beats while allowing for flexible warping (shrinking or expansion) of the beat signal.

Certain aspects provide the determined segmentation to an emotion classification sub-system. The emotion classification sub-system computes heartbeat-based and breathing-based features and uses a support vector machine (SVM) classifier to distinguish various emotional states.

Aspects may have one or more of the following advantages.

Among other advantages, aspects are advantageously able to accurately extract heartbeats from RF reflection signals. Specifically, even errors of 40-50 milliseconds in estimating heartbeat intervals would reduce the emotion recognition accuracy significantly. In contrast, aspects are able to achieve an average error in interbeat-intervals (MI) of 3.2 milliseconds, which is less than 0.4% of the average beat length.

Aspects recognize a subject's emotions by relying on wireless signals reflected off the subject's body.

Aspects recover the entire human heartbeat from RF reflections and can therefore be used in the context of non-invasive health monitoring and diagnosis.

Aspects capture physiological signals without requiring the user to wear any sensors by relying purely on wireless signals reflected off her/his body.

### DESCRIPTION OF THE DRAWINGS

FIG. 1 is a block diagram of an emotion recognition system.

FIG. 2 is a block diagram of a motion signal acquisition module of the system of FIG. 1.

FIG. 3 is an example of a signal representative of a physiological motion of a subject.

FIG. 4 is a block diagram of a motion signal processing module of the system of FIG. 1.

FIG. 5 is an example of a heartbeat component of the signal of FIG. 3.

FIG. 6 is an example of a breathing component of the signal of FIG. 3.

FIG. 7 is a pseudocode description of a heartbeat segmentation algorithm.

FIG. 8 is a segmentation of the heartbeat component of FIG. 5.

FIG. 9 is a heartbeat template determined from the heartbeat component of FIG. 5.

FIG. 10 is a two-dimensional emotion grid.

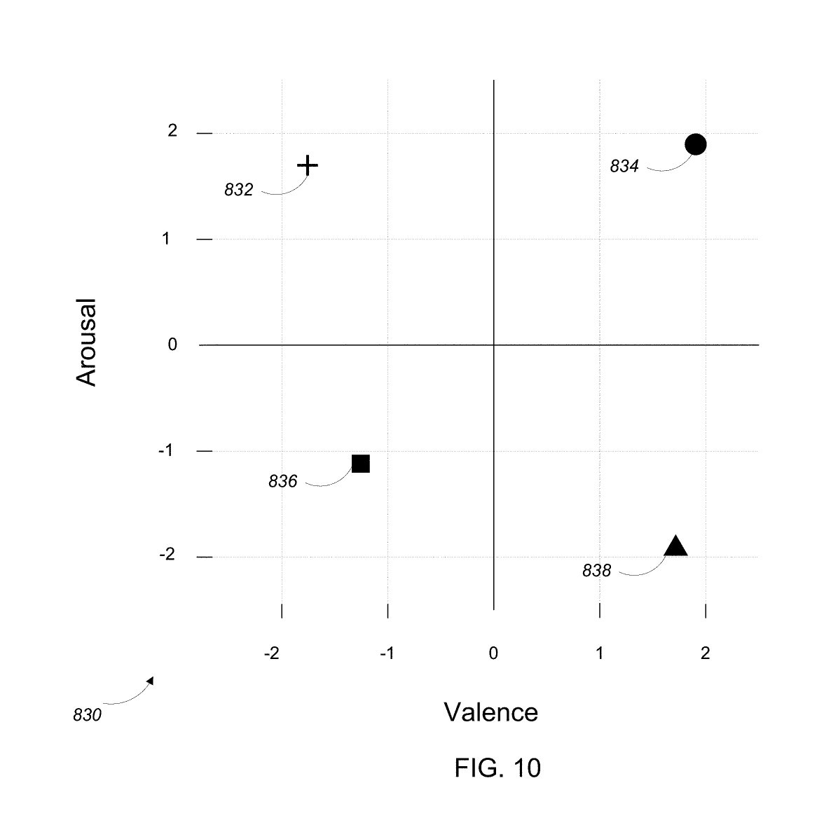

### DETAILED DESCRIPTION

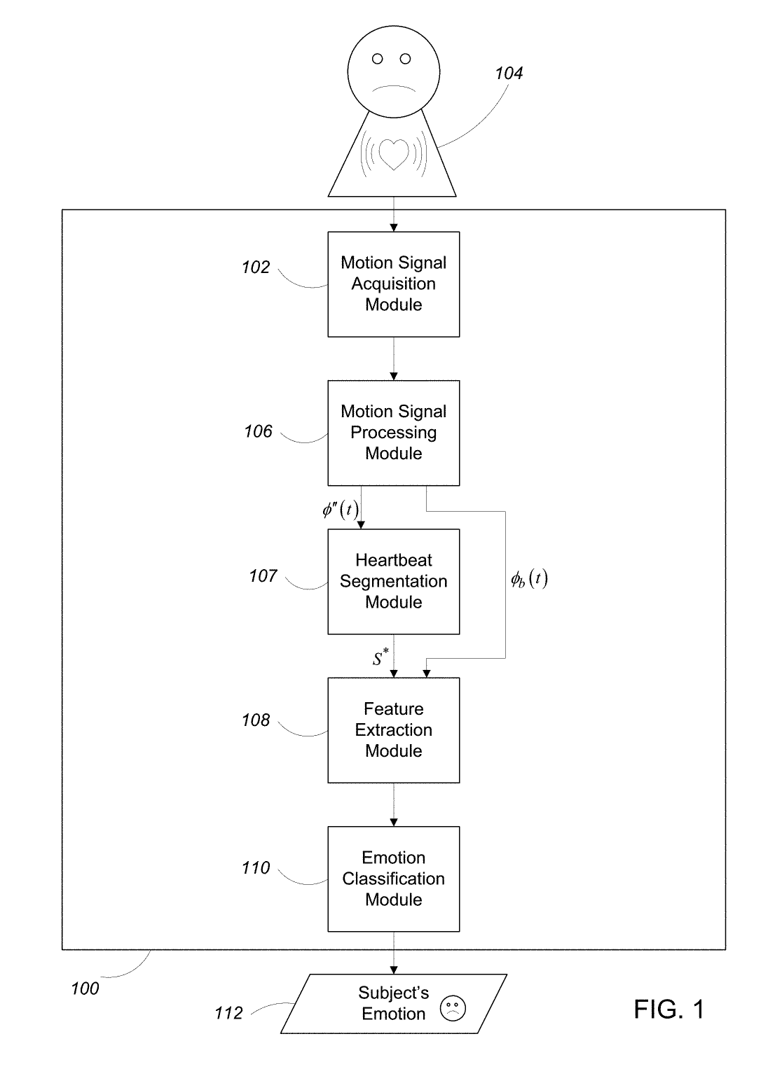

Referring to FIG. 1, an emotion recognition system 100 acquires a signal representative of physiological motion of a subject 104 and processes the acquired signal to infer the subject's emotional state 112. The system 100 includes a motion signal acquisition module 102 for acquisition of signals related to physiological motion of the subject 104, a motion signal processing module 106, a heartbeat segmentation module 107, a feature extraction module 108, and an emotion classification module 110 for classifying the subject's emotional state 112.

### 1 Signal Acquisition

In the example of FIG. 1, the subject's body moves due to both the subject's breathing and the beating of the subject's heart. The motion signal acquisition module 102 includes one or more transducers (not shown) which sense the motion of the subject's body (or any other physiological motion) and generate a signal e.g., an electrical signal) representative of the motion of the subject's body, φ (t).

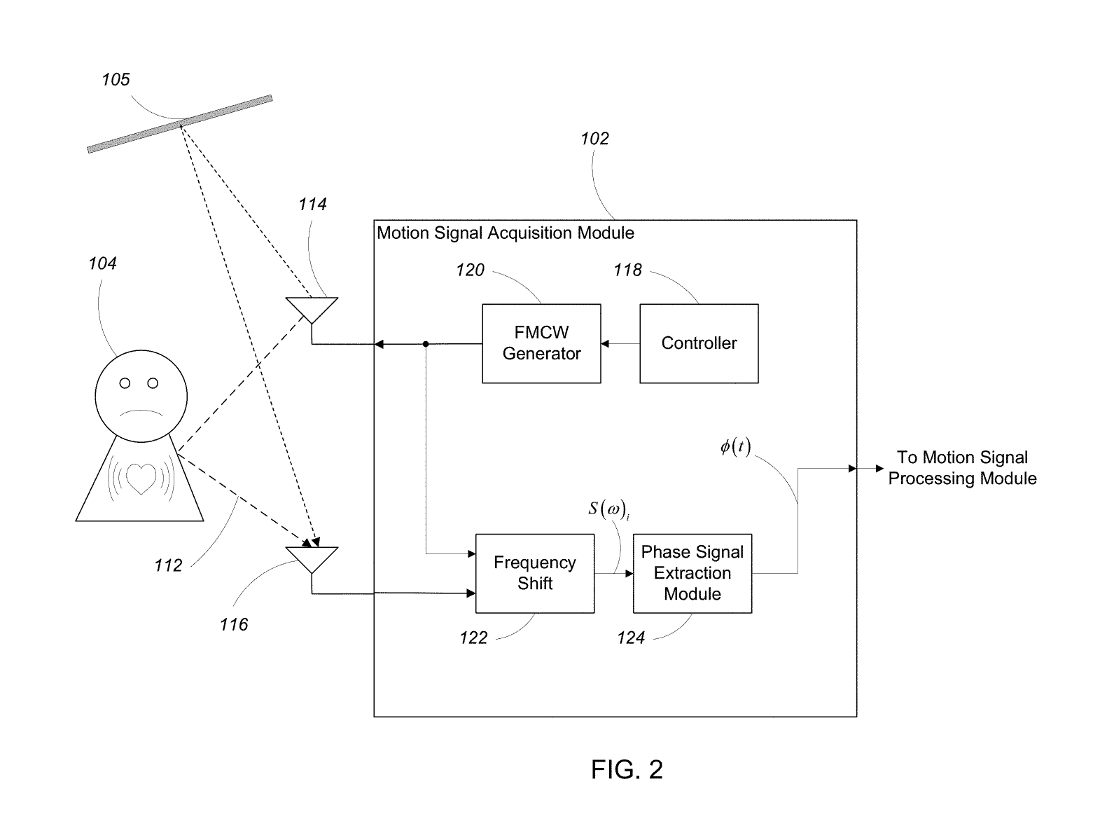

Referring to FIG. 2, in some examples, the motion signal acquisition module 102 uses a wireless sensing technique to generate the signal representative of the motion of the subject's body. Wireless sensing techniques exploit the fact that characteristics of wireless signals are affected by motion in the environment, including chest movements due to inhaling and exhaling and body vibrations due to heartbeats. In particular, wireless sensing systems emit wireless signals which reflect off objects, including the subject 104 in the environment (note that there can be more than one subject in the environment). The reflected signals are then received at the motion sensing acquisition module 102. As the subject 104 in the environment breathes and as their heart beats, a distance traveled by the reflected wireless signals received by the wireless sensing system varies. The wireless sensing system monitors a distance between the antennas of the system and the subject(s) 104 using time-of-flight (TOF) (also referred to as “round-trip time”).

In FIG. 2, the motion signal acquisition module 102 implements a specific wireless sensing technique referred to as Frequency Modulated Continuous Wave (FMCW) wireless sensing. The motion sensing signal acquisition module includes a transmitting antenna 114, a receiving antenna 116, and a number of signal processing components including a controller 118, an FMCW signal generator 120, a frequency shifting module 122, and a phase signal extraction module 124.

In operation, the controller 118 causes the FMCW signal generator 120 to generate repetitions of a signal pattern (e.g., a frequency sweep signal pattern). The repeated signal pattern is provided to the transmitting antenna 114 from which it is transmitted into an environment surrounding the module 102. The transmitted signal reflects off of the one or more subjects 104 and/or other objects 105 such as walls and furniture in the environment and is then received by the receiving antenna 116. The received reflected signal is provided to the frequency shifting module 122 along with the transmitted signal generated by the FMCW signal generator 120. The frequency shifting module 122 frequency shifts (e.g., “downconverts” or “downmixes”) the received signal according to the transmitted signal (e.g., by multiplying the signals) and transforms the frequency shifted received signal to a frequency domain representation (e.g., via a Fast Fourier Transform (FFT)) resulting in a frequency domain representation of the frequency shifted received signal, S (ω)ᵢ at a discrete set of frequencies, ω.

The frequency domain representation of the frequency shifted signal, S (ω) is provided to the phase signal extraction module 124 which processes S (ω)ᵢ to extract one or more phase signals, φ (t). In some examples, the phase signal extraction module 124 processes the frequency shifted signal, S (ω)ᵢ to spatially separate reflections signals from objects and/or subjects in the environment based on their reflection times. In some examples, the phase signal extraction module 124 eliminates reflections from static objects (i.e., objects which do not move over time).

In the example shown in FIG. 2, a path 112 between the transmitting antenna 104 and the receiving antenna 106 is shown reflecting off of a representative subject 104. Assuming a constant signal propagation speed c (i.e., the speed of light), the time of flight (TOF) from a transmitting antenna at coordinates (xₜ, yₜ, zₜ) reflecting from an subject at coordinates (xₒ, yₒ, zₒ) and received at a receiving antenna at coordinates (xᵣ, yᵣ, zᵣ) can be expressed as

$$\frac{1}{c} ( \sqrt{{( {x}_{t} - {x}_{o} )}^{2} + {( {y}_{t} - {y}_{o} )}^{2} + {( {z}_{t} - {z}_{o} )}^{2}} + \sqrt{{( {x}_{r} - {x}_{o} )}^{2} + {( {y}_{r} - {y}_{o} )}^{2} + {( {z}_{r} - {z}_{o} )}^{2}} )$$

In this case, with a single pair of antennas, the TOF associated with the path 112 constrains the location of the subject 104 to lie on an ellipsoid defined by the three-dimensional coordinates of the transmitting and receiving antennas of the path, and the path distance determined from the TOF.

As is noted above, the distance of the ellipsoid from the pair of transmitting and receiving antennas varies with to the subject's chest movements due to inhaling and exhaling and body vibrations due to heartbeats. The varying distance between the antennas 114, 116 and the subject 104 is manifested in the reflected signal as a time varying phase as follows:

$$φ ( t ) = 2 π \frac{d ( t )}{λ}$$

where φ (t) is the phase of the signal, is the wavelength, d (t) is the traveled distance, and t is the time variable. The phase of the signal, φ (t) is output from the motion signal acquisition module 102 as the signal representative of the motion of the subject's body.

Further details of the FMCW based motion sensing techniques described above can be found in PCT Application No. PCT/US2015/027945, titled VITAL SIGNS MONITORING VIA RADIO REFLECTIONS, filed Apr. 28, 2015, and published as WO2015168093, which is incorporated herein by reference.

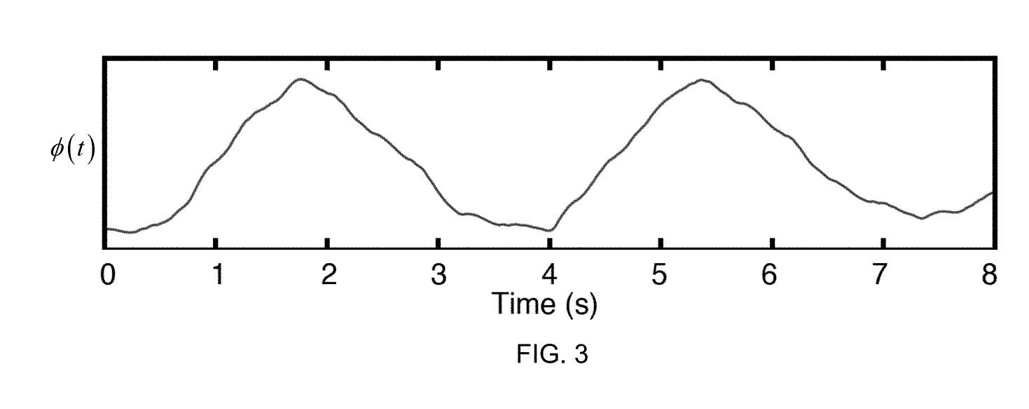

Referring to FIG. 3, one example of the signal representative of the motion of the subject's body, φ (t) acquired by the signal acquisition module 102 has a relatively large breathing component due to the displacement of the subject's chest as they inhale and exhale (i.e., the sinusoidal component with a frequency of ˜0.25 Hz). A heartbeat component of the phase signal manifests as small variations modulating the breathing component, the small variations being caused by minute body vibrations associated with the subject's heartbeating and blood pulsing.

### 2 Motion Signal Processing

Referring again to FIG. 1, the motion signal processing module 106 receives the signal representative of the motion of the subject, φ (t) from the motion signal acquisition module 102 and processes the signal representative of the motion of the subject to separate the heartbeat component, φ″ (t) of the signal from the breathing component, φ_{b} (t) of the signal.

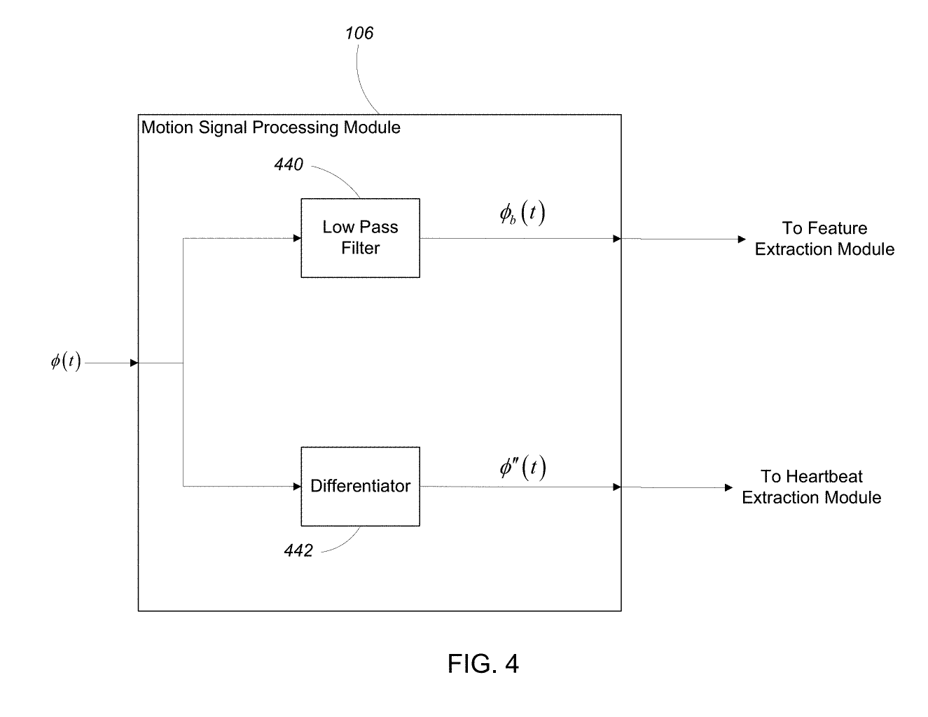

Referring to FIG. 4, the motion signal processing module includes a differentiator 442 which processes the signal representative of the motion of the subject, φ (t) to isolate the heartbeat component, φ″ (t) of the signal and a low pass filter 440 to isolate the breathing component, φ_{b} (t) of the signal.

Since the breathing component is orders of magnitude larger than the heartbeat component, separation of the breathing component from the heartbeat component. To isolate the heartbeat component, φ″ (t) the motion signal processing module 106 leverages the fact that the acceleration of breathing motion is less than that of heartbeat motion. This is because breathing is usually slow and steady while a heartbeat involves rapid contraction of cardiac muscles. Thus, the motion signal processing module 106 includes the differentiator 442 to reduce the effect of the breathing component of the signal relative to the heartbeat component by determining an acceleration signal. In particular, the differentiator 442 computes a second derivative of the signal representative of the motion of the subject, φ″ (t).

In some examples, no analytic expression of φ (t) is available so a numerical method is used to compute the second derivative, φ″ (t). In some examples, due to its robustness to noise, the differentiator 442 implements the following second order differentiator:

$${f}_{0}^{n} = \frac{4 {f}_{0} + ( {f}_{1} + {f}_{- 1} ) - 2 ( {f}_{2} + {f}_{- 2} ) - ( {f}_{3} + {f}_{- 3} )}{16 {h}^{2}}$$

where f₀″ refers to the second derivative at a particular sample, fᵢ refers to the value of the time series i samples away, and h is the time interval between consecutive samples.

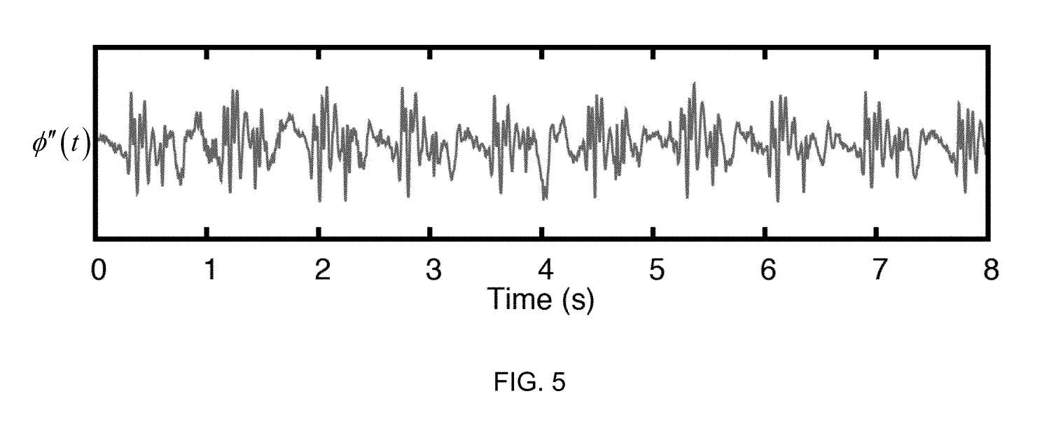

Referring to FIG. 5, one example of an acceleration signal, φ″ (t) output by the differentiator 442 is determined by causing the differentiator 442 to apply the above second order differentiator to the signal representative of the motion of the subject, φ (t). In the resulting acceleration signal, φ″ (t) the signal components due to the heartbeat are prominent due to the acceleration of the motion related to the heartbeat being substantially greater than the acceleration of the motion related to the subject's respiration. In some examples, the motion signal processing module 106 uses a band-pass filter to isolate the signal components related to the heartbeat while also reducing noise present in the signal.

Referring again to FIG. 4, the low pass filter 440 is used to isolate the breathing component, φ_{b} (t) of the signal representative of the motion of the subject, φ (t). In particular, since the breathing component is predominantly low frequency relative to the heartbeat component, the low pass filter can be used to substantially eliminate the heartbeat component from the signal representative of the motion of the subject, φ (t) while leaving the breathing component φ_{b} (t) substantially intact.

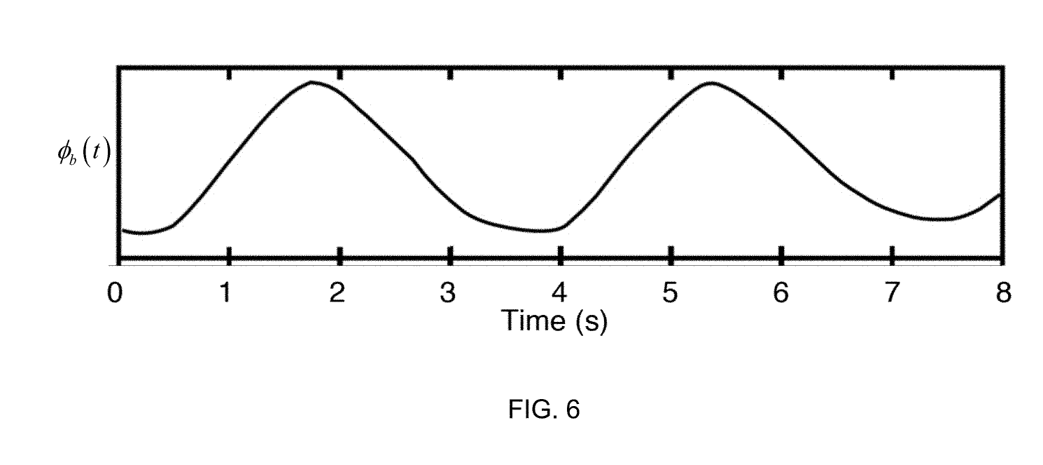

The heartbeat component of the signal (i.e., the acceleration signal), φ″ (t) and the breathing component of the signal, φ_{b} (t) are provided as output from the motion signal processing module 106. Referring to FIG. 6, in one example of a breathing component, φ^{b} (t) output by the low pass filter 440, the relatively higher frequency heartbeat component is substantially removed from the signal representative of the motion of the subject, φ (t), while the breathing component, φ_{b} (t) is substantially intact.

### 3 Heartbeat Segmentation

Referring again to FIG. 1, the heartbeat component of the signal, φ″ (t) is provided to the heartbeat segmentation module 107 which determines an optimal segmentation for the heartbeat component. As is noted above, some approaches to emotion classification utilize small variations in heartbeat intervals of a subject to classify the subject's emotional state. Since the morphology (e.g., the time pattern or shape) of the heartbeats in the heartbeat signal is unknown (due to factors such as the subject's location and posture relative to the system 100), the heartbeat segmentation module 107 uses on an optimization algorithm which jointly determines the morphology of the heartbeats and segments the heartbeats. The resulting segmentation, φ_{S}″ (t) is used to identify, among other features, the small variations in the heartbeat intervals described above.

The optimization algorithm is based on the assumption that successive human heartbeats have the same morphology. That is, while individual heartbeat motions may stretch or compress due to different beat lengths, they will all have the a similar overall shape. With this assumption in mind, the algorithm determines a segmentation that minimizes the differences in shape between heartbeats, while accounting for the fact that the shape of the heartbeats beat is not known a-priori and that the heartbeats may compress or stretch. The algorithm is formulated as an optimization problem over all possible segmentations of the acceleration signal, φ″ (t), as described below.

Given that x=(x₁, x₂, . . . , xₙ) denotes a sequence of length n. A segmentation S={s₁, s₂, . . . } of x is a partition of x into non-overlapping contiguous subsequences (i.e., segments), where each segment sᵢ includes |sᵢ| points. In order to identify each heartbeat, segmentations with segments most similar to one another are identified (i.e., the variation across segments is minimized). Since statistical variance is only defined for scalars or vectors with the same dimension, the definition for vectors with different lengths is extended as such that the variance of segments S={s₁, s₂, . . . } is

$$Var ( S ) = {min}_{μ} {\sum }_{{s}_{i} ∈ S} {‖ {s}_{i} - ω ( μ \vert {s}_{i} \vert ) ‖}^{2}$$

where ω(μ, |sᵢ|) is a linear warping (e.g., through a cubic spline interpolation) of μ into length |sᵢ|.

Note that the above definition is the same as the statistical variance when all the segments have the same length. In the definition above, μ represents the central tendency of all the segments (i.e., a template for the beat shape or morphology).

The algorithm determines an optimal segmentation S* that minimizes the variance of segments, and can be formally stated as follows:

$${S}^{*} = arg {min}_{S} Var ( S )$$

Based on the above statement of the optimal segmentation, the optimization problem can be restated as:

$${minimize}_{S , μ} {\sum }_{{s}_{i} ∈ S} {‖ {s}_{i} - ω ( μ \vert {s}_{i} \vert ) ‖}^{2}$$

subject to

bₘᵢₙ≦|sᵢ|≦bₘₐₓ,sᵢεS

where bₘᵢₙ and bₘₐₓ are constraints on the length of each heartbeat cycle.

The optimization problem attempts to determine the optimal segmentation S and template (i.e., morphology) μ that minimize the sum of the square differences between segments and template. This optimization problem involves both combinatorial optimization over S and numerical optimization over μ. Exhaustively searching all possible segmentations has exponential complexity.

To avoid this exponential complexity, the algorithm alternates between updating the segmentation and template rather than estimating the segmentation S and the template μ simultaneously. During each iteration, the algorithm updates the segmentation given the current template and then updates the template given the new segmentation. For each of these two sub-problems, the algorithm obtains global optimal with linear time complexity.

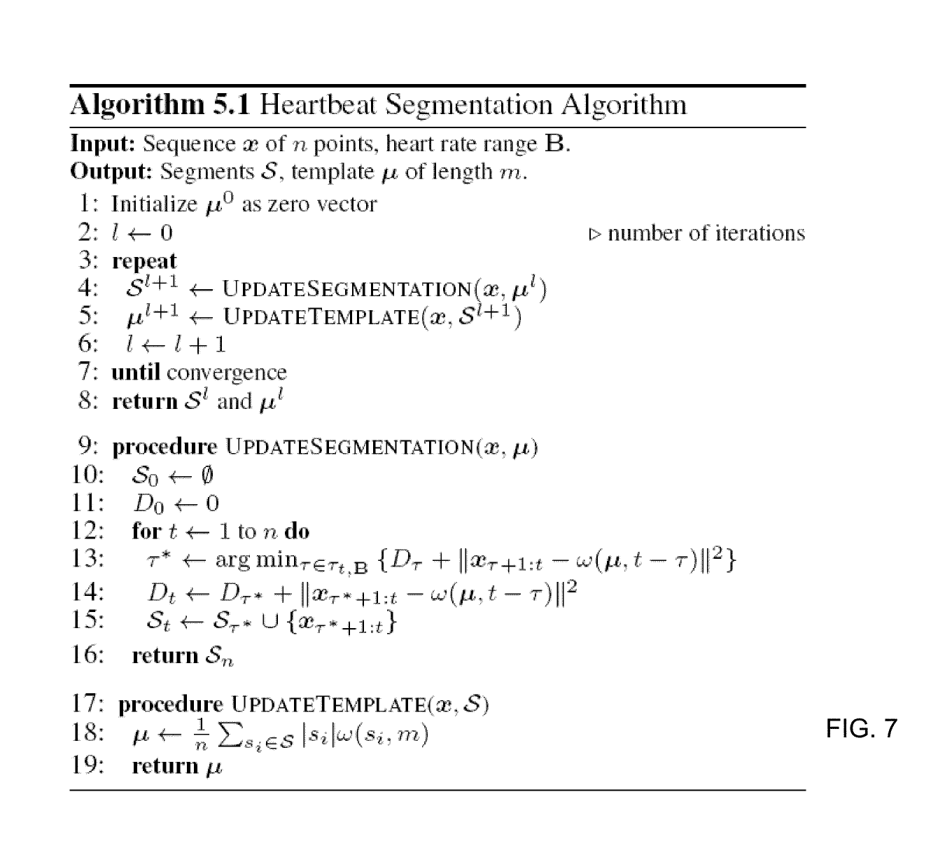

Referring to FIG. 7, a pseudocode description of the heartbeat segmentation algorithm receives as input a sequence, x of n data samples and an allowable heart rate range, B. The heartbeat segmentation algorithm generates an output including a number of segments, S and a template μ of length m.

In Line 1 of the pseudocode description, a vector representing μ is initialized to include all zeroes. In Line 2 of the pseudocode description, a number of iterations, l is initialized to zero. In Lines 3-7 of the pseudocode description a loop executes in which the segmentation, S and the template, μ are iteratively updated until the algorithm converges. In particular, in Line 4 of the pseudocode description an updated segmentation, S^{l+1} is determined by invoking an UPDATESEGMENTATION procedure on the sequence, x of data samples and the most recently updated version of the template, μ^{l}. In Line 5 of the pseudocode description an updated version of the template, μ^{l+1} is determined by invoking an UPDATETEMPLATE procedure on the sequence, x of data samples and the most recently updated version of the segmentation, S^{l+1}. In Line 5 of the pseudocode description the number of iterations, l is incremented. The UPDATESEGMENTATION and the UPDATETEMPLATE procedures are repeatedly called until the algorithm converges. Once the algorithm converges, the final segmentation, S^{l} and the final template, μ^{l} are returned in Line 8 of the pseudocode description.

Referring to Lines 9-16 of the psuedocode description, the UPDATESEGMENTATION procedure receives as input a sequence, x of n data samples and a template, μ. The procedure returns an n^{th} segmentation, Sₙ which is determined as follows:

$${S}_{n} = arg {min}_{S} {\sum }_{{s}_{i} ∈ S} {‖ {s}_{i} - ω ( {μ}^{l} , \vert {s}_{i} \vert ) ‖}^{2}$$

Though the number of possible segmentations grows exponentially with the length of x, the above optimization problem is solved efficiently using dynamic programming. The recursive relationship for the dynamic program is as follows: if Dₜ denotes the minimal cost of segmenting sequence x_{1:t}, then:

$${D}_{t} = {min}_{τ ∈ {τ}_{t : B}} \{ {D}_{τ} + {‖ {x}_{τ + 1 : t} - ω ( μ , t - τ ) ‖}^{2} \}$$

where τ_{t,B} specifies possible choices of τ based on segment length constraints. The time complexity of the dynamic program based on Eqn. 6 is O (n) and the global optimum is guaranteed.

Referring to Lines 17-19 of the pseudocode description, the UPDATETEMPLATE procedure receives as input a sequence, x of n data samples and a segmentation, S. The procedure returns an updated template, μ. The updated template is determined as:

$$\begin{matrix}{μ}^{l + 1} = arg {min}_{μ} {\sum }_{{s}_{i} ∈ {S}^{l + 1}} {‖ {s}_{i} - ω ( μ \vert {s}_{i} \vert ) ‖}^{2} \\ = arg {min}_{μ} {\sum }_{{s}_{i} ∈ {S}^{l + 1}} \vert {s}_{i} \vert \cdot {‖ μ - ω ( {s}_{i} \vert m \vert ) ‖}^{2}\end{matrix}$$

where m is the required length of template. The above optimization problem is a weighted least squares with the following closed-form solution:

$${μ}^{l + 1} = \frac{{\sum }_{{s}_{i} ∈ {S}^{l + 1}} \vert {s}_{i} \vert ω ( {s}_{i} , m )}{{\sum }_{{s}_{i} ∈ {S}^{l + 1}} \vert {s}_{i} \vert} = \frac{1}{n} {\sum }_{{s}_{i} ∈ {S}^{l + 1}} \vert {s}_{i} \vert ω ( {s}_{i} , m )$$

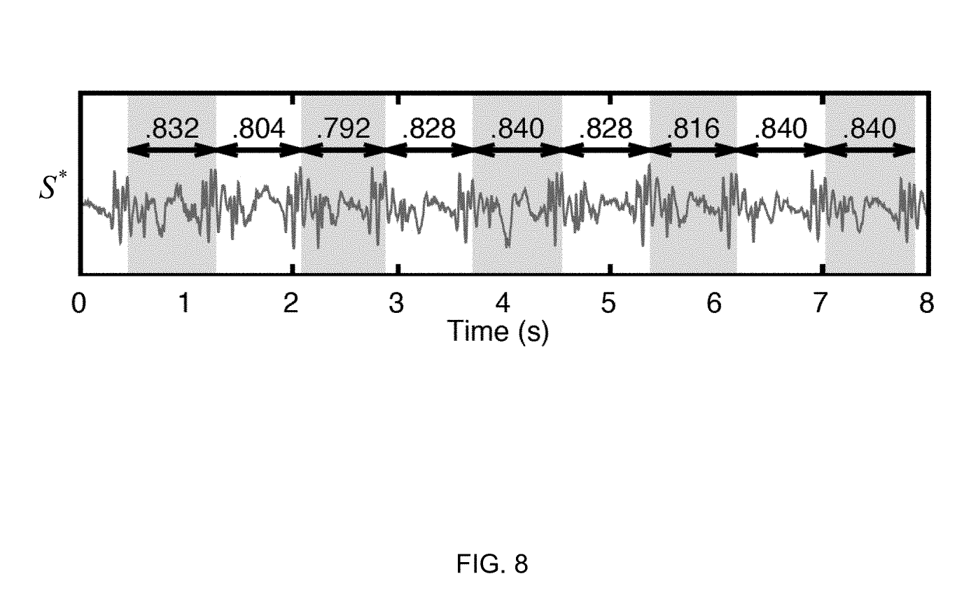

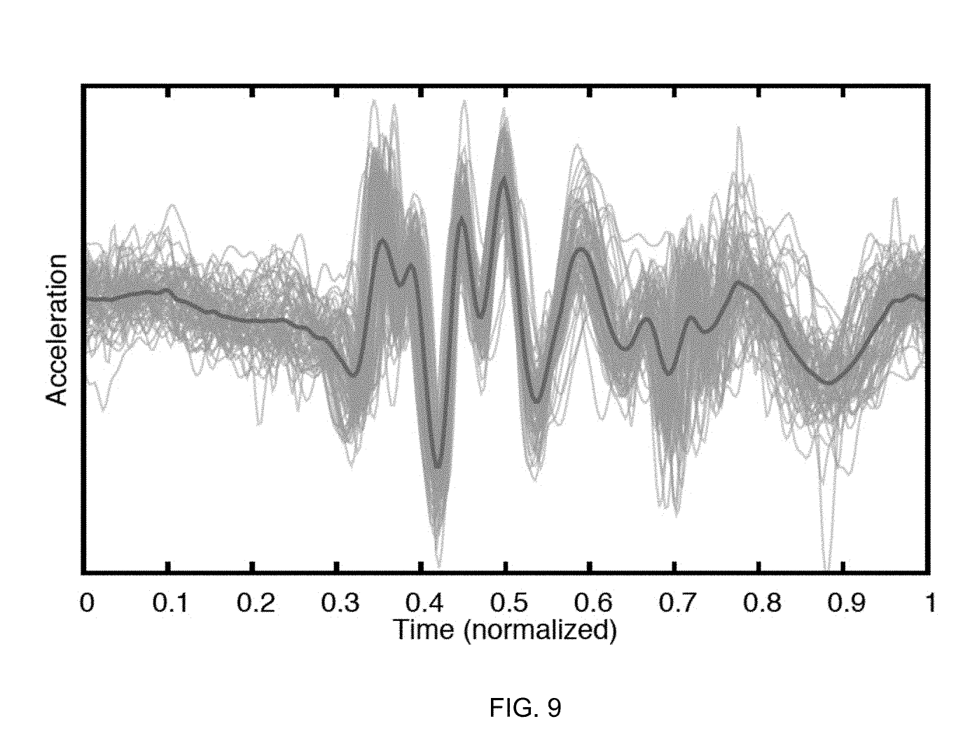

Referring to FIG. 8, the result of applying the above-described algorithm to the acceleration signal is a segmented acceleration signal, S*. Referring to FIG. 9, a heartbeat morphology discovered from the acceleration signal by the above-described algorithm is shown.

### 4 Feature Extraction

The segmented acceleration signal and the respiration signal are provided to the feature extraction module 108 which determines features for use by the emotion classification module 110 using the determined morphology and segmentation of the heartbeat signal and the respiration signal.

In some examples, the feature extraction module 108 extracts features in the time domain such as the Mean, Median, SDNN, PNN50, RMSSD, SDNNi, meanRate, sdRate, HRVTi, and TINN. In some examples, the feature extraction module 108 extracts features in the frequency domain such as Welch PSD (LF/HF, peakLF, peakHF), BurgPSD (LF/HF, peakLF, peakHF), Lomb-Scargle PSD: LF/HF, peakLF, peakHF). In some examples, the feature extaction module 108 extracts Poincare features such as SD₁, SD₂, SD₂/SD₁. In some examples, the feature extraction module 108 extracts nonlinear features such as SampEn₁, SampEn₂, DFAₐ₁₁, DFA₁, and DFA₂.

In some examples, the feature extraction module 108 extracts breathing features such as the irregularity of breathing. To do so, the feature extraction module 108 identifies each breathing cycle by peak detection in the breathing component, φ_{b} (t). It then uses some or all of the features described above to measure the variability of breathing.

### 5 Emotion Classification

Referring again to FIG. 1, the features extracted by the feature extraction module 108 are provided to the emotion classification module 110 which processes the features according to, for example, an emotion model to generate a classification of the subject's emotion 112.

In some examples, the emotion classification module 110 implements an emotion model which has a valence axis and an arousal axis. Very generally, the emotion model classifies between four basic emotional states: Sadness (negative valence and negative arousal), Anger (negative valence and positive arousal), Pleasure (positive valence and negative arousal), and Joy (positive valence and positive arousal). For example, referring to FIG. 8, a 2D emotion grid 830 includes a number of exemplary emotion classification results generated by an emotion model. A first emotion classification result 832 has a positive arousal value and a negative valence and therefore signifies a subject with an angry emotional state. A second emotion classification result 834 has a positive arousal value and a positive valence value and therefore signifies a subject with a joyous emotional state. A third emotion classification result 836 has a negative arousal value and a negative valence value and therefore signifies a subject with a sad emotional state. A fourth emotion classification result 838 has a negative arousal value and a positive valence value and therefore signifies a subject with a pleasurable emotional state.

In some examples, the emotion model of the emotion classification module 110 is trained to classify the subject's emotion into the 2D emotion grid using a set of training data. In some examples, the set of training data includes a number of sets of features measured from a number of subjects, with each set of features being associated with a known emotional state in the 2D emotion grid. The emotion classification module 110 uses machine learning techniques to analyze the training data and to train the emotion model (e.g., a support vector machine (SVM) classifier model) based on statistical relationships between sets of features and emotional states. Once the emotion model is trained, the emotion classification module 110 is able to receive extracted features for a subject from the feature extraction module 108 and to predict an emotion of the subject by applying the emotion model to the extracted features. Further details related to emotion classification systems and methods can be found in, for example: J. Kim and E. André. “Emotion recognition based on physiological changes in music listening. Pattern Analysis and Machine Intelligence,” IEEE Transactions on, 30(12):2067-2083, 2008 and P. J. Lang. “The emotion probe: studies of motivation and attention.” American psychologist, 50(5):372, 1995, the contents of which are incorporated herein by reference.

In some examples, the features extracted by the feature extraction module 108 differ from one subject to another for the same emotional state. Further, those features could be different for the same subject on different days. Such variations may be caused by multiple factors, including caffeine intake, sleep, and baseline mood of the day. In order to ensure that the model is user-independent and time-independent, the emotion classification module 110 incorporates a baseline emotional state: neutral. That is, the emotion classification module 110 leverages changes of physiological features instead of absolute values. Thus, in some examples, the emotion classification module 110 calibrates the computed features by subtracting for each feature its corresponding values calculated at the neutral state for a given person on a given day. This calibration may incorporated into the emotion model used by the emotion classification module 110 and/or may be part of a pre-processing step applied to the extracted features before they are supplied to the emotion model.

In some examples, using all of the features listed above with a limited amount of training data can lead to over-fitting. For this reason, in some examples, the emotion classification module 110 selects a set of features that is most relevant to emotions. This selection not only reduces the amount of data needed for training but also improves the classification accuracy on the test data. In some examples, the emotion classification module 110 learns which features best contribute to the accuracy of the emotion model while training the emotion model. In some examples, this learning is accomplished using an 11-SVM which selects a subset of relevant features while training the emotion model.

### 6 Alternatives

It is noted that, while the embodiment described above uses contact-less RF sensing to sense motion of the subject's body (e.g., skin or internal structures, or clothing covering the skin), in other examples, the signal acquisition module 102 uses accelerometers coupled to the subject's body (either directly or via clothing or wearable accessories on the subject's body) to sense the motion of the subject's body. In yet other examples, the signal acquisition module 102 uses ultrasound measurement techniques to sense motion (e.g., motion of blood in the subject's vasculature). It should be appreciated that any number of other suitable approaches can be used to sense the motion related to the subject's physiology. In general, the motion signal acquisition module 102 conditions the signal representative of the motion of the subject's body by, for example, filtering, amplifying, and sampling the signal such that signal output by the motion signal acquisition module 102 is usable by the downstream modules of the system 100.

The system described above employs an FMCW wireless sensing technique which includes transmitting repetitions of a single signal pattern (e.g., a frequency sweep signal pattern). However, it is noted that in some examples, the system performs repeated transmissions with each transmission including a different signal pattern (which is a priori known to the system). For example, each transmission may include an a priori known pseudo-random noise signal pattern. Since each signal pattern is a priori known to the system, the system can determine information such as time of flight by comparing the transmitted a priori known signal to a received reflection of the transmitted signal (e.g., by cross-correlation of the known signal and the received reflection of the transmitted signal).

It is noted that the signal representative of physiological motion can represent any number of different types of physiological motion. For example, the signal can represent physiological motion at the macro scale such as movement of a subject's skin. The signal can also represent physiological motion at a smaller scale such as movement of blood through a subject's vasculature. For example, a video recording (i.e., a recording captured using a video camera) of a subject can be analyzed to identify small changes in coloration of the subject's skin due to movement of blood into and out of the vasculature in and adjacent to the subject's skin. The observed changes in coloration of the subject's skin can then be used to infer the subject's emotion.

In some examples, the system is configured to determine a cognitive state (e.g., a degree of confusion, distraction, attentiveness, etc) of a subject using a cognitive state classifier (e.g., a support vector machine based cognitive state classifier). The cognitive state classifier classifies the subject's cognitive state based at least in part on the determined segmentations of the heartbeat components of the motion based physiological signals associated with the subject.

In some examples, features of the subject's heartbeat are extracted from the heartbeat components of the motion based physiological signals associated with the subject and are mapped to cardiac functions. In some examples the features include one or more of peaks, valleys, and inflection points in the heartbeat components.

### 7 Implementations

Systems that implement the techniques described above can be implemented in software, in firmware, in digital electronic circuitry, or in computer hardware, or in combinations of them. The system can include a computer program product tangibly embodied in a machine-readable storage device for execution by a programmable processor, and method steps can be performed by a programmable processor executing a program of instructions to perform functions by operating on input data and generating output. The system can be implemented in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

It is to be understood that the foregoing description is intended to illustrate and not to limit the scope of the invention, which is defined by the scope of the appended claims. Other embodiments are within the scope of the following claims.

What is claimed is:

1. A method for processing motion based physiological signals representing motion of a subject using signal reflections from the subject, the method comprising:
emitting a radio frequency transmitted signal comprising one or more transmitted signal patterns from a transmitting element;
receiving, at one or more receiving elements, a radio frequency received signal comprising a combination of a plurality of reflections of the transmitted signal, at least some reflections of the plurality of reflections of the transmitted signal being associated with the subject;
processing time successive patterns of reflections of the transmitted signal patterns to form the one or more motion based physiological signals including, for at least some reflections of the of the plurality of reflections, forming a motion based physiological signal representing physiological motion of a subject from a variation over time of the reflection of the transmitted signal in the received signal; and
processing each motion based physiological signal of a subset of the one or more motion based physiological signals to determine a segmentation of a heartbeat component of the motion based physiological signal, the processing including
determining the heartbeat component,
determining a template time pattern for heartbeats in the heartbeat component, and
determining a segmentation of the heartbeat component based on the determined template time pattern.

2. The method of claim 1 wherein the transmitted signal is a frequency modulated continuous wave (FMCW) signal including repetitions of a single signal pattern.

3. The method of claim 1 wherein the one or more transmitted signal patterns include one or more pseudo random noise sequences.

4. The method of claim 1 wherein determining the heartbeat component includes mitigating an effect of respiration on the motion based physiological signal including determining a second derivative of the motion based physiological signal.

5. The method of claim 1 wherein determining the heartbeat component includes mitigating an effect of respiration on the motion based physiological signal including filtering the motion based physiological signal using a band pass filter.

6. The method of claim 1 wherein determining the template time pattern for heartbeats in the heartbeat component and determining the segmentation of the heartbeat component including jointly optimizing the time pattern for the heartbeats and the segmentation of the heartbeat component.

7. The method of claim 1 further comprising determining a cognitive state of the subject based at least in part on the determined segmentation of the heartbeat component of the motion based physiological signal associated with the subject.

8. The method of claim 7 wherein the cognitive state of the subject includes one or more of a state of confusion, a state of distraction, and a state of attention.

9. The method of claim 1 further comprising determining an emotional state of the subject based at least in part on the determined segmentations of the heartbeat components of the motion based physiological signals associated with the subject.

10. The method of claim 9 wherein determining the emotional state of the subject is further based on respiration components of the one or more motion based physiological signals.

11. The method of claim 9 further comprising determining the respiration components of the one or more motion based physiological signals including applying a low-pass filter to the one or more motion based physiological signals.

12. The method of claim 9 wherein determining the emotional state of the subject includes applying an emotion classifier to one or more features determined from the determined segmentations of the heartbeat components of the motion based physiological signals.

13. The method of claim 10 wherein determining the emotional state of the subject includes applying an emotion classifier to one or more features determined from the determined segmentations of the heartbeat components of the motion based physiological signals and to one or more features determined from the respiration components of the one or more motion based physiological signals.

14. The method of claim 9 further comprising presenting the emotional state in a two-dimensional grid including a first, arousal dimension and a second, valence dimension.

15. The method of claim 1 wherein the motion based physiological signal represents physiological motion of a subject from a variation over time of a phase angle of the reflection of the transmitted signal in the received signal.

16. The method of claim 1 further comprising extracting features from the heartbeat components of each of the motion based physiological signals and mapping the extracted features to one or more cardiac functions, the features including as peaks, valleys, of inflection points.

17. A method for determining an emotional state of a subject, the method comprising:
receiving the motion based physiological signal associated with a subject, the motion based physiological signal including a component related to the subject's vital signs; and
determining an emotional state of the subject based at least in part on the component related to the subject's vital signs.

18. The method of claim 17 wherein the component related to the subject's vital signs includes a periodic component, the method further comprising determining a segmentation of the periodic component.

19. The method of claim 18 wherein determining the segmentation of the periodic component includes determining a template time pattern for periods in the periodic component and determining the segmentation of the periodic component based on the determined template time pattern.

20. The method of claim 18 wherein determining the emotional state of the subject is based at least in part on the segmentation of the periodic component.

21. The method of claim 18 wherein the periodic component includes at least one of a heartbeat component and a respiration component.

22. The method of claim 21 further comprising determining the heartbeat component including determining a second derivative of the motion based physiological signal.

23. The method of claim 21 further comprising determining the heartbeat component including applying a band-pass filter to the motion based physiological signal.

24. The method of claim 21 further comprising determining the respiration component including applying a low-pass filter to the motion based physiological signal.

25. The method of claim 17 wherein determining the emotional state of the subject includes applying an emotion classifier to one or more features determined from the motion based physiological signal associated with the subject.

26. The method of claim 20 wherein determining the emotional state of the subject includes applying an emotion classifier to one or more features determined from the determined segmentation of the periodic component.

27. The method of claim 17 further comprising presenting the emotional state in a two-dimensional grid including a first, arousal dimension and a second, valence dimension.

28. The method of claim 17 wherein the motion based physiological signal associated with the subject is associated with an accelerometer measurement.

29. The method of claim 17 wherein the motion based physiological signal associated with the subject is associated with an ultrasound measurement.

30. The method of claim 17 wherein the motion based physiological signal associated with the subject is associated with a radio frequency based measurement.

31. The method of claim 17 wherein the motion based physiological signal associated with the subject is associated with a video based measurement.
